(19) United States
(12) Patent Application Publication
(10) Pub. No.: US 2026/0356988 A1
(43) Pub. Date: Nov. 12, 2026

(54) NEW CARBOXYMETHYL CELLULOSE AND METHOD OF PREPARATION

(71) Applicant: DANISCO USA INC., New Century, KS (US)

(72) Inventor: Oliver Petermann, Bomlitz (DE)

(21) Appl. No.: 19/140,094

(22) PCT Filed: Jan. 16, 2024
(86) PCT No.: PCT/US2024/011610

§ 371 Date: Jun. 17, 2025

(30) Foreign Application Priority Data
Jan. 16, 2023 (EP) 23151794.7

(51) Int. Cl. C08B 11/12

(52) U.S. Cl. CPC C08B 11/12

(57) ABSTRACT

This invention relates to a new process for preparing carboxymethyl (CMC), new types of carboxymethyl cellulose, their methods of preparation and uses.

### FIELD OF THE INVENTION

The present invention is directed to a new process for preparing carboxymethyl (CMC), new types of carboxymethyl cellulose (CMC), their methods of preparation and uses.

### BACKGROUND

CMCs are used in a wide variety of technical fields. Areas which benefit from the thickening and gel-forming properties of CMC are, for example, oil industry (e.g. in drilling fluids), food industry, pharmaceutical industry, paper industry, electrical and electronic industry, textile industry and construction industry.

Enteric coating agents usually have a carboxylic functionality which renders the polymer soluble at high pH and insoluble at low pH. CMC has carboxylic carboxymethyl groups which should make CMC insoluble at low pH too, but due to the high pKa value CMC is not completely insoluble at low pH. Therefore, the use of CMC as an enteric coating agent in prescribed drugs (Rx) and nutraceutical formulations has been limited. Coatings for tablets which as an ingredient as CMC has been described in U.S. Pat. No. 4,547,571A.

CMC with low degree of substitution (DS) is of interest due to its potential biodegradable, enteric, associative thickening and thixotropic properties. CMC with a DS lower than 0.4 is known to be insoluble in water according to literature (James N. BeMiller, in Carbohydrate Chemistry for Food Scientists (Third Edition), 2019, 223-240). Preparation of CMCs having a low degree of substitution (DS) by alkalization with sodium hydroxide (NaOH) or potassium hydroxide (KOH) and subsequent carboxymethylation with monochloroacetic acid is described in numerous documents, e.g. WO 2011/120533.

However, there is still the need to prepare CMC with a low degree of substitution (DS) having a high solution quality, such as low turbidity.

### OBJECT OF INVENTION

In order to overcome the current challenges of providing CMC with a low degree of substitution (DS) having a high solution quality, such as low turbidity, the inventors have shown that using more than 2.5 mol of alkalizing agent in a process of preparing carboxymethyl cellulose provides a CMC with a low DS and high solution quality.

The inventors have also found that the present CMC's having a low or very low DS may be used as an enteric coating agent. This is surprising as a skilled person in the art would have expected that the more carboxymethyl groups are present in the CMC (high DS), the better enteric properties the CMC would show, and not the opposite. The low DS CMC fulfills the U.S. Pharmacopeial Convention (USP) requirements for delayed drug release depending on the thickness of the coating. Otherwise, it fulfills the requirement for nutraceutical formulation (film is stable over 1 h in HCl).

It is therefore an object of the present invention to provide a new process for preparing types of CMC with a low degree of substitution (DS) having a high solution quality and uses thereof. It is further an object of the present invention to provide novel CMC's. It is further an object of the present invention to provide the use of the present CMC's as a modified release coating.

### SUMMARY OF THE INVENTION

The problem is solved by a process of preparing carboxymethyl cellulose (CMC) comprising the steps of:
a) suspending non-regenerated cellulose powder in a solvent/water mixture comprising water and isopropyl alcohol and adding an alkalizing agent either as a solid or as an aqueous solution, and reacting the non-regenerated cellulose with the alkalizing agent in the presence of isopropyl alcohol and water; and
b) then adding monohaloacetic acid or a salt thereof to the alkalized cellulose of step a), and reacting the alkalized cellulose of step a) with the monohaloacetic acid or a salt thereof,
   wherein more than 2.5 mol of alkalizing agent is used in step a) and wherein from 0.2 to 2 mol of monohaloacetic acid or a salt thereof is used in step b), each based on 1 mol of anhydroglucose unit of the cellulose.

The present invention is also directed to the novel CMCs obtained and obtainable by this process.

The present invention further concerns CMCs having a degree of substitution equal to or below 0.25, preferably equal to or below 0.2, more preferably equal to or below 0.19 and a turbidity equal to or below 1500 NTU, preferably equal to or below 1200 NTU, more preferably equal to or below 700 NTU, most preferably equal to or below 500 NTU, measured as a 2 weight % aqueous solution.

Another aspect of the present invention is the use of the CMCs according to the present invention as modified release coatings.

A further aspect of the present invention is the use of the CMCs according to the present invention in various technical fields where they can act as thickening agent and/or gelling agent, binder, stabilizer, emulsifier, film forming agent, suspending agent, protective colloid, crystallization inhibitor, or as enteric coatings.

### DESCRIPTION OF DRAWINGS

FIG. 1: The graph shows APAP (N-acetyl-para-aminophenol or paracetamol) diffusion through a 1 cm² CMC film in simulated gastric fluid (0.1N HCl) at 37 °C. APAP concentration was 2 mg/ml and maximum drug diffusion of 0.2 mg/ml after 2 h indicates pass of USP test for delayed release. Drug release was higher than 0.2 mg/ml, but the film stayed intact indicating suitability for neutraceuticals.

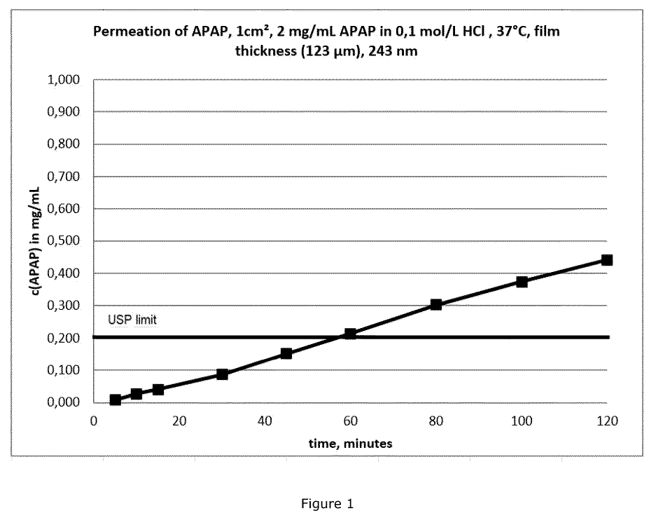

FIG. 2: Permeation of APAP showing that drug release was higher than 0.2 mg/ml, but the film stayed intact indicating suitability for neutraceuticals. Drug release was slower than for FIG. 1 due to higher film thickness.

FIG. 3: Permeation of APAP showing that drug release was not higher than 0.2 mg/ml indicating suitability for pharmaceuticals.

### DETAILED DESCRIPTION OF INVENTION

The detailed aspects of this invention are set out below. In part some of the detailed aspects are discussed in separate sections. This is for ease of reference and is in no way limiting. All of the embodiments described below are equally applicable to all aspects of the present invention unless the context specifically dictates otherwise.

The term “CMC” or “carboxymethyl cellulose” as used herein encompasses cellulose substituted with groups of the formula —CH₂CO₂A, wherein A is hydrogen or a monovalent cation, such as K⁺ or preferably Na⁺.

The process of preparing carboxymethyl cellulose according to the present invention comprises reacting non-regenerated cellulose with an alkalization agent and monohaloacetic acid or a salt thereof, preferably the sodium salt.

As used herein, the term “regenerated cellulose” refers to cellulose that has been prepared by regeneration (i.e., return to solid form) from a solution that includes dissolved cellulose fibers. The term “non-regenerated cellulose” refers to cellulose that has not gone through a process of dissolving cellulose, typically cellulose fibers, in a solvent, and then regenerating it from that solution, i.e., recovering cellulose in solid form. In particular “non-regenerated cellulose” refers to cellulose that has not been recovered from a cellulose xanthogenate solution or a cellulose/cupramonium solution or a solution of cellulose in N-methylmorpholin-N-oxide.

The alkalizing agent is preferably NaOH, KOH or a mixture thereof. In one embodiment of the invention NaOH is used as the alkalization agent. In another embodiment KOH is used as the alkalization agent. In the latter case remaining K⁺ ions in the produced CMC are preferably exchanged by Na⁺ ions in a subsequent step.

The monohaloacetic acid can be used as free acid or in its salt form. Preferably, monochloroacetic acid or its salt is used in the inventive process, including all preferred embodiments described in the following.

According to the present invention, it is important to observe the molar ratios of each of the above-described components during preparation of the CMC.

The alkalizing agent, including the preferred alkalizing agents described above, is used in an amount from 2.5 to 10 mol, preferably in an amount from 3.1 to 10 mol, more preferably in an amount from 3.5 to 8 mol, most preferably in an amount from 4 to 7 mol; based on 1 mol of anhydroglucose unit of the cellulose.

The monohaloacetic acid, preferably monochloroacetic acid, is used in an amount of from 0.2 to 2 mol of monohaloacetic acid or a salt thereof, preferably in an amount from 0.3 to 1.5 mol, more preferably in an amount from 0.4 to 1 mol, more preferably in an amount from 0.5 to 0.8 mol; each based on 1 mol of anhydroglucose unit of the cellulose. These preferred amounts of monohaloacetic acid are preferably used in combination with the preferred amounts of alkalizing agent indicated above.

The ratio alkalizing agent/monohaloacetic acid is preferably between 2.4-20, more preferably between 2.6-20; most preferably between 2.8-20. The ratio alkalizing agent/monohaloacetic acid is further preferably between 3.0-15; more preferably between 3.5-14 and most preferably between 4.0-13.

Water is used in the alkalizing step (a) in an amount of preferably less than 15 moles of water, based on 1 mol of anhydroglucose unit of the cellulose. In another embodiment, water is used in the alkalizing step (a) in an amount of preferably more than or equal to 15 moles of water, based on 1 mol of anhydroglucose unit of the cellulose. In another embodiment, water is used in the alkalizing step (a) in an amount of preferably from 10 to 14.95 mol, more preferably in an amount from 12 to 14.92, most preferably in an amount from 14 to 14.9 mol, based on 1 mol of anhydroglucose unit of the cellulose. In another embodiment, water is used in the alkalizing step (a) in an amount of preferably from 15 to 45 mol, more preferably in an amount from 15.5 to 45 mol, more preferably in an amount from 20 to 45, most preferably in an amount from 30 to 45 mol, based on 1 mol of anhydroglucose unit of the cellulose.

These preferred amounts of water are preferably used in combination with the preferred amounts of alkalizing agent and monohaloacetic acid indicated above. Isopropyl alcohol is generally used in the alkalizing step (a) in an amount of from 100 to 10 mol, preferably from 80 to 20 mol, more preferably from 60 to 30 mol, and most preferably from 55 to 32 mol, each based on 1 mol of anhydroglucose unit of the cellulose. These preferred amounts of isopropyl alcohol are preferably used in combination with the preferred amounts of alkalizing agent, monohaloacetic acid and water indicated above.

In a preferred embodiment of the invention non-regenerated cellulose is reacted with the alkalization agent in the presence of water and isopropyl alcohol and an additional amount of methanol. Methanol, if present, is preferably used in the alkalizing step (a) in an amount of from 10 to 1 mol, more preferably from 9 to 2 mol, even more preferably from 8 to 3 mol, and most preferably from 7 to 4 mol, each based on 1 mol of anhydroglucose unit of the cellulose. These preferred amounts of methanol are preferably used in combination with the preferred amounts of alkalizing agent, monohaloacetic acid, water, and isopropyl alcohol indicated above.

It is understood that the solvents (i.e. water, isopropyl alcohol and optionally methanol) introduced in the alkalizing step (a) are typically not removed deliberately before or during reaction of the alkalized cellulose with the monohaloacetic acid and thus said solvents also constitute the reaction medium of step (b). However, it is noted that the amounts of water, isopropyl alcohol and optional methanol specified above only refer to the amounts present during alkalization in step (b) and do not include any additional amounts of said solvents that may optionally be added later in the process. e.g. during reaction with monohaloacetic acid in step (b).

In some embodiments, including embodiments using the preferred amounts of components as indicated above, the alkalizing agent and the monohaloacetic acid are used in a molar ratio of from 20 to 2.5, more preferably from 15 to 2.8, even more preferably from 12 to 3, and most preferably of about 11 to 4.

The inventive process of preparing CMCs as described above leads to new types of CMCs.

Typically, the novel CMCs according to the present invention have a degree of substitution equal to or below 0.25, preferably equal to or below 0.2, more preferably equal to or below 0.19 and a turbidity equal to or below 1500 NTU, preferably equal to or below 1200 NTU such as equal to or below 1000 NTU, more preferably equal to or below 700 NTU, most preferably equal to or below 500 NTU, measured as a 2 weight % aqueous solution.

The degree of substitution is the average number of OH groups that have been substituted in one anhydroglucose unit. It is determined according to ASTM D 1439-03 “Standard Test Methods for Sodium Carboxymethylcellulose; Degree of Etherification, Test Method B: Nonaqueous Titration”. The treatment of a solid sample of the CMC with glacial acetic acid at boiling temperature releases an acetate ion quantity equivalent to the sodium carboxymethyl groups. These acetate ions can be titrated as a strong base in anhydrous acetic acid using a perchloric acid standard solution. The titration end point is determined potentiometrically. Other alkaline salts of carboxylic acids (e. g. sodium glycolate and di-sodium diglycolate) behave similarly and are co-titrated.

It is quite surprising that the new types of CMC according to the present invention form highly viscous solutions or gels in aqueous media despite their low to moderate degree of substitution.

In one aspect of the present invention the novel CMCs, including those having preferred degrees of substitution as described above, typically have a viscosity, measured as a 2 weight % aqueous solution as described below, of at least 100 mPa·s, preferably at least 440 mPa·s, more preferably at least 600 m·Pas, and most preferably of at least 800 mPa·s. The viscosity is generally up to 30,000 m·Pas, preferably up to 25,000 m·Pas, more preferably up to 20,000 m·Pas, measured as a 1 weight % aqueous solution as described below.

In order to determine the viscosity a 2% solution is prepared as follows (total amount of solution 300 g). 294 g deionized water (water in CMC is subtracted) is placed in 500 ml screw cap bottle which is being thermostated at 25 °C. 6 g (dry weight) of the CMC is then stirred in evenly at a rotating speed of approx. 1300 to prepare a solution. After that the solution is stirred at a constant rotating speed (approx. 1000-1500 rpm) and at a temperature of 25 °C for 1 h and 30 min. Then the stirrer is switched off, the solution is kept at 25 °C without stirring for 30 minutes before the viscosity is determined. The viscosity of the solution was measured using a Brookfield LVT viscometer, 30 rpm, 25° with spindle 3 for example A, spindle 2 for example B, spindle 3 for examples C, D, E and spindle 1 for examples F, G and H.

Not only that aqueous solutions of the present CMCs are highly viscous, it is further very surprised that the clarity of the aqueous CMC solutions is good, i.e., much better than it would have been expected based on the relatively low degree of substitution.

In another aspect of the present invention the novel CMCs, including those preferred CMCs described above, wherein the carboxymethyl cellulose has a degree of substitution equal to or below 0.25, preferably equal to or below 0.2, more preferably equal to or below 0.19 and a turbidity equal to or below 1500 NTU, preferably equal to or below 1200 NTU such as equal to or below 1000 NTU, more preferably equal to or below 700 NTU, most preferably equal to or below 500 NTU, measured as a 2 weight % aqueous solution.

Preferably, 2 weight % aqueous solutions of the novel CMCs according to the present invention, including those preferred CMCs described above, have transmission values of from 100 to 5%, more preferably from 100 to 5%, even more preferably from 100 to 8%, measured as indicated below.

2 weight % aqueous solutions of CMC are prepared as described above in connection with the viscosity analysis with additional rolling on a roller device for 24 h. The transmission and the turbidity are analyzed with the Turbidimeter 2100AN using a tungsten filament lamp emitting light of a wavelength of 300 to 1000 nm (Hach Company, Loveland, Colorado, USA). The transmission is the photometric analysis of the transmitted light through a sample cell (diameter: 24 mm) at a given wavelength (here 610 nm). It is given in % transmitted light. The system is calibrated against water (100% transmission). The result is the average of eight measurements. The turbidity is the analysis of the scattered light through a sample cell (diameter: 24 mm) and is given in NTUs (nephelometric turbidity units) according to USEPA method 180.1. The analysis is performed against a formazin standard ranging from <0.1 NTU to 7500 NTU (StablCal™, catalogue number 2659505). A USEPA method 180.1 filter module (catalogue number 3031200, 400 to 600 nm) is used. The result is the average of ten measurements.

Within the meaning of the present application the terms “high viscosity CMC” or “CMC having high or low viscosity” or similar expressions refer to the viscosities of aqueous solutions of the CMC.

The type of non-regenerated cellulose to be used for preparing the CMC is not essential for the present invention and is governed by the intended end-use of the CMC. Conventional non-regenerated starting materials are natural cellulose, such as cotton linters and wood pulp, e.g. hard wood or soft wood pulp. Typically, cotton linters or wood pulp are used, depending on the desired application of the CMC. Pulps according to present invention have a limiting viscosity number measured according to ISO 5351:2010 from 50 to 300 mL/g, preferably from 1000 to 3000 mL/g and more preferably from 300 to 1000 mL/g.

Thanks to the present invention it is now also possible to prepare food grade CMCs derived from wood pulp which can be used as powerful thickening agents in the food industry due to their superior viscosity and gel forming properties. Although wood pulp is the preferred raw material in the present process for economic reasons, it is, of course, also possible to employ cotton linters if a CMC of extraordinarily high viscosity is desired for some reasons.

The essential steps of the preparation method of carboxymethyl cellulose are alkalizing the cellulose by reaction with the alkalizing agent (alkalizing step (a)) and adding monohaloacetic acid to cause etherification of the alkali cellulose (carboxymethylation step (b)).

The cellulose is first alkalized in the presence of water and isopropyl alcohol and optionally methanol in the alkalizing step (a) and then the monohaloacetic acid is added in the carboxymethylation step. In the alkalizing step, the single components (e.g. cellulose, water, isopropyl alcohol, alkalizing agent, and optionally methanol) can be added in arbitrary order. However, it is preferred that the cellulose is combined with water, isopropyl alcohol, and optionally methanol in a first step and the alkalizing agent is then added in a subsequent step. If methanol is used, it can be added to the reaction mixture before, at the same time or shortly after the addition of the monohaloacetic acid, but preferably it is added at the same time as water and/or isopropyl alcohol, either separately or as a mixture with water and/or isopropyl alcohol.

There are various options how to contact the cellulose with the alkalizing agent. Preferably, cellulose powder is used as starting material. Typically, the cellulose powder is suspended (slurried) in the solvent/water mixture comprising water, isopropyl alcohol and optionally methanol before the alkalizing agent is added as a solid or as an aqueous solution. However, the total amount of water used in the alkalizing step should be in an amount of less than 15 moles of water, based on 1 mol of anhydroglucose unit of the cellulose. In another embodiment, water is in an amount of more than or equal to 15 moles of water, based on 1 mol of anhydroglucose unit of the cellulose. In another embodiment, water is in an amount of from 10 to 14.95 mol, more preferably in an amount from 12 to 14.92, most preferably in an amount from 14 to 14.9 mol, based on 1 mol of anhydroglucose unit of the cellulose. In another embodiment, water is in an amount of from 15 to 45 mol, more preferably in an amount from 15.5 to 45 mol, more preferably in an amount from 20 to 45, most preferably in an amount from 30 to 45 mol, based on 1 mol of anhydroglucose unit of the cellulose. The amount of cellulose in the starting slurry of cellulose in the solvent/water mixture is preferably within the range of from 1 to 14 weight %, more preferably from 3 to 12 weight %, and most preferably from 4 to 10 weight %, each based on the total weight of cellulose and solvent/water mixture.

According to the preferred slurry method, the alkalizing step (a) is typically conducted at a temperature within the range of from 10 to 40 °C, preferably from 15 to 40 °C, more preferably from 15 to 30 °C, even more preferably from 18 to 25 °C, and most preferably at about 20 °C. Typical reaction times for the alkalization step range from 15 to 120 min, preferably from 40 to 100 min, and more preferably from 50 to 80 min depending on the reaction temperature. In one embodiment the alkalizing step is conducted at about 20 °C for 50 to 70 min, preferably for 60 min.

In the carboxymethylation step (b) the monohaloacetic acid or a salt thereof, preferably the sodium salt, can be added neat or as a solution, preferably as an aqueous solution or as a solution in water and isopropyl alcohol and optionally methanol. The carboxymethylation step is typically conducted at a temperature within the range of from 40 to 100 °C, preferably from 50 to 90 °C, more preferably from 60 to 80 °C, and most preferably at about 70 °C. In preferred embodiments the monohaloacetic acid is already added to the reaction mixture before the carboxymethylation temperature is reached; more preferably the monohaloacetic acid is already added before the heating-up phase begins or within the first minutes of the heating-up phase, generally at a temperature which is described above for the alkalizing step, e.g. at from 15 to 40 °C. The early addition of the monohaloacetic acid at lower temperatures may avoid an undesired degradation of the cellulose (resulting in a decrease of viscosity) which may occur at the higher temperatures in absence of monohaloacetic acid. After the desired temperature of the carboxymethylation step (carboxymethylation temperature) as mentioned before is reached, the carboxymethylation temperature is typically held for a period of from 0 to 180 min, preferably from 20 to 140 min.

The product obtained from the carboxymethylation step, i.e. the etherification, is the sodium or potassium salt of carboxymethyl cellulose depending on which alkalizing agent was employed.

According to a preferred embodiment the CMC salt is neutralized by adding acid, preferably an organic acid such as acetic acid or formic acid, more preferably acetic acid. Herein “neutralizing” means adjusting the pH value to a value of from 6 to 9, preferably 7 to 8. The CMC is then separated from the reaction mixture and purified, if necessary, depending on the intended end-use, and dried. Purification is performed according to standard methods well-known to the person skilled in the art. For example, the CMC can be washed with organic solvents including mixtures of organic solvents and solvent(s)/water mixtures, such as methanol, acetone, methanol/water mixtures, acetone/methanol mixtures, and methanol/isopropyl alcohol/water mixtures.

The outstanding property of the CMCs according to the present invention is their ability to form highly viscous aqueous solutions or gels having a low turbidity in combination with a low to moderate degree of substitution of the cellulose. Furthermore, the CMCs according to the present invention having a DS below 0.5 such as a DS between 0.4-0.5 are also biodegradable contrary to the current commercially available CMCs.

It has been found that the use of KOH as alkalizing agent in the present process results in CMCs with even superior gel-forming properties (higher viscosity and clarity) as compared to the use of NaOH. The improved gel-forming character of the CMC is maintained even if the K⁺ ions in the produced CMC have been exchanged by Na⁺ ions in a subsequent step. K⁺ ions in the produced CMC can be exchanged by Na⁺ ions after separating the CMC from the reaction mixture by, for example, washing it extensively with methanol comprising about 20 weight percent of a saturated aqueous sodium chloride solution. After the exchange of the K⁺ ions by Na⁺ ions the CMC can be purified according to known procedures, for example as described above or in the examples below.

The CMCs according to the present invention are promising candidates for a lot of different applications. For example, they can be used as thickening agents and/or gelling agents, binders, stabilizers, emulsifiers, film forming agents, suspending agents, protective colloids, or crystallization inhibitors. Their beneficial properties make the present CMCs useful in a variety of technical fields, including their use in drilling fluids, such as oil drilling fluids; in pharmaceutical dosage forms, such as capsules, tablets, solutions, suspensions, emulsions, creams, and lotions; in food products, such as processed meat products, dairy products, bakery products, structured, extruded or coated food products, like meat substitutes, dressings, sauces, marinades, soups and spreads; in cosmetic applications, such as hair styling products, toothpastes, emulsions, creams, and lotions; in detergents; in paper coating or paper binding; in welding electrodes; in textile applications, such as textile printing and dyeing; in ceramic applications, such as clay bodies and tile production; in adhesives; in paints; and in construction applications, such as gypsum or cement based dry mortar applications.

While some dosage forms are effective if released immediately into the blood stream, it may be advantageous to either release the active ingredient slowly or target the release to a specific area of the body. The CMCs according to the present invention are promising candidates for use as modified release coatings of solid dosage forms. The modified release may be a delayed release or an extended release.

The following examples are provided for illustrative purposes only and are not intended to limit the scope of the claims that follow. Unless otherwise indicated, all parts and percentages are by weight.

### EXAMPLES

The following examples are provided for illustrative purposes only and are not intended to limit the scope of the claims that follow. Unless otherwise indicated, all parts and percentages are by weight.

### Cellulose Starting Material

For examples A, B and C milled cellulose wood pulp with an intrinsic viscosity of 660 ml/g was used (Biofloc 96 from Tartas). For examples D and E milled cotton pulp with an intrinsic viscosity of 1740 ml/g was used (PCS 2400 from Gaomi). For examples F, G and H milled wood pulp with an intrinsic viscosity of 410 ml/g was used (Cellunier F from Rayonier)

In all of the examples 129.7 g of dry cellulose (corresponds to 0.8 mol anhydroglucose units (AGU)) were used together with varying amounts of isopropyl alcohol (IPA), methanol (MeOH), water, NaOH or KOH, and monochloroacetic acid (MCA) as indicated in the Table. MCA was added as a 80% solution in water.

First, the cellulose powder was introduced into a dry 3 litre laboratory reactor and then, the solvent mixture of isopropyl alcohol, methanol and water was added under a nitrogen atmosphere. NaOH (as prills) or KOH (as prills) were added under stirring at a temperature of 20 °C, stirring was continued for 15 minutes after which hydrogen peroxide was added in a concentration of 3% in water (examples D-H); stirring continued at 20 °C for a total of 60 min. Afterwards, the reaction mixture was heated up to 70 °C within 40 min and the monochloroacetic acid was added as a 80 weight % aqueous solution during the first minutes of the heating-up phase. The temperature of 70 °C was held for 120 min during the carboxymethylation step and then the reaction mixture was cooled down to 20 °C within about 60 min. The pH value of the cooled reaction mixture was adjusted to 7.5 with acetic acid and the solution was subsequently filtered. The filter residue was subsequently washed at least 5 times with 2 | of a isopropanol/methanol/water mixture (isopropanol/methanol/water weight ratio of 5:3:2) until no chloride ions were detected in the filtrate. This was followed by one final washing step with pure methanol. The washed samples were dried overnight in the drying cabinet at 55 °C and then milled in a laboratory mill.

The degree of substitution of the CMCs obtained was determined as described in the general part of the specification. 2 weight % solutions of the CMC samples were also prepared, and their viscosity, turbidity and transmission were determined as described in the general part of the specification. The results are given in the below Table.

| | | H₂O₂ | MeOH¹ | H₂O¹ | NaOH | MCA | | | | | |
|---|---|---|---|---|---|---|---|---|---|---|---|
| | IPA¹ | [mol/ | [mol/ | [mol/ | [mol/ | [mol/ | | | | | Trans- |
| | [mol/ | mol | mol | mol | mol | mol | NaOH/ | | Viscosity | Turbidity | mission |
| Sample | mol AGU] | AGU] | AGU] | AGU] | AGU] | AGU] | MCA ratio | DS² | [mPa · s] | [NTU] | [%] |
| (A) | 50.15 | — | 5.48 | 36.6 | 6.32 | 0.63 | 10.03 | 0.19 | 1760 | 683 | 9 |
| (B) | 48.65 | — | 5.45 | 42.65 | 6.32 | 0.63 | 10.03 | 0.16 | 120 | 1104 | 6 |
| (C) | 51.65 | — | 5.51 | 32.55 | 6.32 | 0.63 | 10.0 | 0.22 | 2620 | 333 | 16 |
| (D) | 38.28 | 0.014 | 4.08 | 14.89 | 4.2 | 0.7 | 6.1 | 0.4 | 2440 | 3 | 97 |
| (E) | 38.28 | 0.014 | 4.08 | 14.73 | 4.2 | 0.6 | 6.1 | 0.34 | 1800 | 3 | 96 |
| (F) | 34.21 | 0.165 | 3.53 | 15.19 | 3.0 | 0.6 | 5 | 0.33 | 18 | 5 | 94 |
| (G) | 34.61 | 0.33 | 3.54 | 15.19 | 3.2 | 0.6 | 5.3 | 0.33 | 7.5 | 8 | 89 |
| (H) | 35.23 | 0.165 | 3.55 | 11.88 | 3.0 | 1.0 | 3 | 0.6 | 8 | 2 | 97 |

¹amounts used in the alkalizing step
²degree of substitution

Two of the above CMC syntheses, sample D and E, were conducted with water concentrations below 15 mol/mol AGU and high concentrations of sodium hydroxide (>4 mol/mol AGU) resulting in CMC having a DS below 0.5. The solutions from these CMCs show a very low turbidity and high transmittance. The synthesis was conducted as described on page 11 of WO 2011/120533 except that the amounts of reactants and solvents according to the table above was used. Sample D has a DS of 0.4 and a turbidity of 3.1 NTU which is a lower turbidity than the CMC of example 5 from WO 2011/120533 which has a DS of 0.4 and a turbidity of 14 NTU. Sample E has a DS of 0.34 and a turbidity of 3 NTU which is a lower turbidity than the CMC of example 18 from WO 2011/120533 which has a DS of 0.36 and a turbidity of 10 NTU.

The turbidity of the samples of WO 2011/120533 were measured at a lower concentration (1%) than the turbidity of samples D/E which were measured at 2%. The higher the concentration the higher is the turbidity of the solution. Nevertheless, even at 2% concentration sample D/E show a lower turbidity than the prior art.

The CMC syntheses for samples A, B and C resulted in CMC's having a DS of 0.19, 0.16 and 0.22, respectively. All solutions were turbid but viscous. Films were casted several times on a glass plate from a 2% solution and dried at RT. Films casted from the solutions were homogenous, firm and elastic showing that the CMC's are still functional. The film contained a lot of fibers which didn't prevent film formation or have a negative impact on film properties. The films were a homogenous film and not of non-woven type. A piece of the film of sample A (123 μm) was put into a Franz cell diffusion cell and the paracetamol (APAP) diffusion through was measured during 2 h at 37 °C in HCl. The film stays stable in gastric fluids and allow sustained drug release over time.

Sample H is a CMC with a DS of 0.6 being synthesized with high concentrations of alkali but with water concentration <15 mol/mol AGU.

A film from sample H (165 μm thickness) disintegrates within 15 min in the Franz cell in HCl.

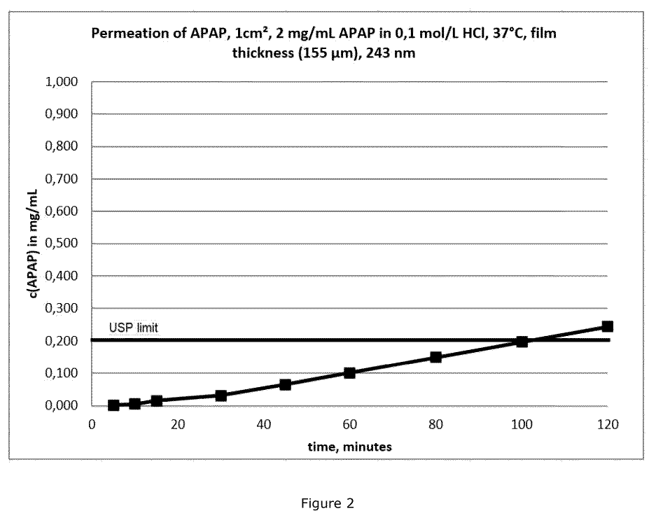

Sample F is a CMC with a DS of 0.33 being synthesized with high concentrations of alkali but with water concentration >15 mol/mol AGU. A film from sample F casted from a 4% solution (155 μm thickness) stays intact in the Franz cell. Acid resistance doesn't fulfill USP requirements, but nutraceutical requirements. See FIG. 2. A slightly higher film thickness should be sufficient to ensure fulfilment of USP requirements as shown for sample G (sample G see table above).

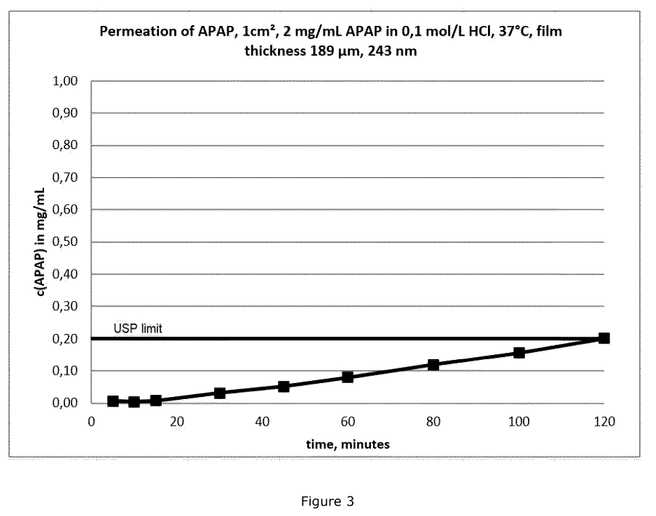

The sample G has a DS of 0.33 being synthesized with high concentrations of alkali but with water concentration >15 mol/mol AGU. Films were casted several times on a glass plate from a 7.5% solution and dried at RT. A piece of the film (1 cm×1 cm) film (189 μm thickness) stays intact in the Franz cell. Drug release after 2 h was 0.2 mg of APAP which is the USP limit. The film passed the USP test. See FIG. 3.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A process of preparing carboxymethyl cellulose comprising the steps of:
a) suspending non-regenerated cellulose powder in a solvent/water mixture comprising water and isopropyl alcohol and adding an alkalizing agent either as a solid or as an aqueous solution, and reacting the non-regenerated cellulose with the alkalizing agent in the presence of isopropyl alcohol and water; and
b) then adding monohaloacetic acid or a salt thereof to the alkalized cellulose of step a), and reacting the alkalized cellulose of step a) with the monohaloacetic acid or a salt thereof,
wherein more than 2.5 mol of alkalizing agent is used in step a) and wherein from 0.2 to 2 mol of monohaloacetic acid or a salt thereof is used in step b); each based on 1 mol of anhydroglucose unit of the cellulose.

2. The process according to claim 1, wherein the ratio of alkalizing agent/monohaloacetic acid is between 2.4-20, preferably between 2.6-20; more preferably between 2.8-20.

3. The process according to claim 2, wherein the ratio of alkalizing agent/monohaloacetic acid is between 3.0-15; more preferably between 3.5-14 and most preferably between 4.0-13.

4. The process according to claim 1, wherein step a) is conducted in the presence of water, isopropyl alcohol and methanol.

5. The process according to claim 1, wherein from 100 to 10 mol of isopropyl alcohol are used in step a) based on 1 mol of anhydroglucose unit of the cellulose.

6. The process according to claim 1, wherein said process is conducted in the presence of less than 15 moles of water based on 1 mol of anhydroglucose unit of the cellulose.

7. The process according to claim 1, wherein said process is conducted in the presence of more than or equal to 15 moles of water based on 1 mol of anhydroglucose unit of the cellulose.

8. The process according to claim 1, wherein the process is conducted in the presence of water in an amount from 10 to 14.95 mol, more preferably in an amount from 12 to 14.92, most preferably in an amount from 14 to 14.9 mol; each based on 1 mol of anhydroglucose unit of the cellulose.

9. The process according to claim 1, wherein the process is conducted in the presence of water in an amount from 15 to 45 mol, more preferably in an amount from 15.5 to 45 mol, more preferably in an amount from 20 to 45, most preferably in an amount from 30 to 45 mol; each based on 1 mol of anhydroglucose unit of the cellulose.

10. The process according to claim 1, wherein the alkalizing agent is used in an amount from 2.5 to 10 mol, preferably in an amount from 3.1 to 10 mol, more preferably in an amount from 3.5 to 8 mol, most preferably in an amount from 4 to 7 mol; each based on 1 mol of anhydroglucose unit of the cellulose.

11. The process according to claim 1, wherein the alkalizing agent is sodium hydroxide (NaOH), potassium hydroxide (KOH) or a mixture thereof, preferably sodium hydroxide.

12. The process according to claim 11, wherein when KOH is used as the alkalization agent and the K⁺ ions are exchanged by Na⁺ ions in a subsequent step.

13. The process according to claim 1, wherein the monohaloacetic acid or a salt thereof is used in an amount from 0.3 to 1.5 mol, preferably in an amount from 0.4 to 1 mol, more preferably in an amount from 0.5 to 0.8 mol; each based on 1 mol of anhydroglucose unit of the cellulose.

14. The process according to claim 1, wherein the monohaloacetic acid or a salt thereof is monochloroacetic acid or its salt.

15. The process according to claim 1, wherein the alkalizing agent is added as a solid.

16. The process according to claim 1, wherein the product obtained after alkalization and reaction with monohaloacetic acid or a salt thereof is neutralized by adding an acid, preferably acetic acid.

17. Carboxymethyl cellulose obtained or obtainable by the process of claim 1.

18. A carboxymethyl cellulose, wherein the carboxymethyl cellulose has a degree of substitution equal to or below 0.25, preferably equal to or below 0.2, more preferably equal to or below 0.19 and a turbidity equal to or below 1500 NTU, preferably equal to or below 1200 NTU, more preferably equal to or below 700 NTU, most preferably equal to or below 500 NTU, measured as a 2 weight % aqueous solution.

19. (canceled)

20. (canceled)

21. (canceled)

22. (canceled)

23. (canceled)
